(19) United States
(12) Patent Application Publication
(10) Pub. No.: US 2026/0216204 A1
(43) Pub. Date: Jul. 30, 2026

(54) METHODS FOR TREATING LOWER RISK MYELODYSPLASTIC SYNDROME

(71) Applicant: Constellation Pharmaceuticals, Inc., Boston, MA (US)

(72) Inventors: Katarina Luptakova, Newton, MA (US); Stefan Haertle, Planegg (DE)

(21) Appl. No.: 19/141,429

(22) PCT Filed: Dec. 19, 2023
(86) PCT No.: PCT/US2023/084959

§ 371 Date: Jun. 20, 2025

Related U.S. Application Data
(60) Provisional application No. 63/433,833, filed on Dec. 20, 2022.

(51) Int. Cl. A61K 31/55; A61P 35/00

(52) U.S. Cl. CPC A61K 31/55; A61P 35/00

(57) ABSTRACT

The present disclosure relates to the use of pelabresib, and pharmaceutically acceptable salts and hydrates thereof, for treating lower risk myelodysplastic syndrome (LR-MDS) and conditions associated therewith.

### RELATED APPLICATIONS

This application claims the benefit of priority to U.S. Provisional Application No. 63/433,833, filed Dec. 20, 2022, the entire contents of which are incorporated herein by reference.

### BACKGROUND

Myelodysplastic syndrome (MDS) is a heterogeneous collection of clonal marrow stem-cell disorders characterized by ineffective hematopoiesis leading to blood cytopenias, with a third of patients progressing eventually to acute myeloid leukemia (AML). More than 80% of MDS patients carry more than 1 known recurrently mutated genes at the time of diagnosis (Haferlach et al., Leukemia. 2014; 28: 241-7). Most cases of MDS are genomically complex, with one dominant clone and many clones containing multiple cooperating mutations that can contribute to disease progression and/or relapse. Besides acquired somatic mutations, cytokine aberrations, and immune dysregulation, alternations in the bone marrow microenvironment also play integral roles in the pathogenesis.

The abnormalities in the microenvironment result in disruption of the integrity of normal hematopoiesis, leading to an increased apoptotic index, aberrant cellular biology, and dysplasia of bone marrow progenitor cells. An abnormal microenvironment may also function as a milieu for selective expansion of the MDS clone and lead to disease progression. Dysregulation of key regulators in bone marrow stem and progenitor cells leads to abnormal hematopoiesis, apoptosis, and cell proliferation leading to an increased risk of infections, bleeding, and progression to AML (Ganan-Gomez et al., Leukemia. 2015; 29: 1458-69).

MDS is classified by risk according to the International Prognostic Scoring System (IPSS), the original edition and the Revised IPSS (IPSS-R). See e.g., Greenberg et al., Blood 1997; 89: 2079-88 and Greenberg et al., Blood 2012; 120: 2454-65. Approximately 80% of lower risk MDS patients have anemia and transfusion dependency is this population is associated with inferior survival. In patients ≥60 years of age, chronic anemia is associated with several co-morbidities, including cardiovascular complications, a higher risk of falls and bone fractures, and shorter survival. Even without transforming to AML, MDS is often fatal due to the associated complications related to cytopenias and infections.

There are limited treatment options available for lower risk MDS (LR-MDS), particularly those who have anemia and are transfusion dependent. Typically, the first line of treatment for lower risk MDS is erythropoiesis-stimulating agents (ESAs), which target early erythropoiesis and increasing proliferation. However, red blood cell (RBC) transfusion-dependent MDS patients often have a decreased response to ESAs. Luspatercept, an activin ligand trap that reduces aberrant Smad 2/3 signaling, recently received approval by the US Food and Drug Administration (FDA) for treatment of anemia for adult patients with very low- to intermediate-risk MDS with ring sideroblasts or with myelodysplastic/myeloproliferative neoplasm with ring sideroblasts and thrombocytosis who failed ESA treatment and require ≥2 RBC units over 8 weeks. Luspatercept, however, is not indicated for other MDS subtypes, and it may not be suitable for patients with several common co-morbidities, including thromboembolism and hypertension. Furthermore, the majority of patients treated with luspatercept did not achieve the desired response. Lenalidomide is another treatment option for lower risk MDS patients; however, it is only approved for patients with a del 5q cytogenic abnormality. Currently, the only known cure for MDS is bone marrow transplantation. This intensive treatment, however, is only reserved for eligible patients and those who have higher risk MDS.

Additional therapeutic options to treat MDS, and its associated conditions, are therefore needed.

### BRIEF DESCRIPTION OF THE DRAWINGS

FIG. 1 shows IL-6 protein release of primary MDS patient PBMCs stimulated with LPS in parallel to pelabresib treatment determined via an electrochemiluminescence-based immunoassay.

FIG. 2 shows the percentage of megakaryocyte-erythroid CD34+ progenitors (left) and percentage of CD34− pro-erythroblast/erythroblast (right) in isolated hematopoietic stem cell samples treated with erythroid expansion supplement alone (DMSO only) or treated with erythroid expansion supplement and 250 nM of pelabresib, n=2 donors.

FIG. 3 shows cytokine release of primary MDS patient PBMCs stimulated with LPS in parallel to pelabresib treatment determined via an electrochemiluminescence-based immunoassay. Values were normalized to “DMSO only” control, n=7-10 donors, meant SEM

### SUMMARY

LR-MDS is characterized by excessive apoptosis in the bone marrow and an autoimmune disease-like profile. In addition, multiple genetic abnormalities that are common in MDS, such as TET2 and SF3B1 mutations, are capable of inducing NF-kB and pro-inflammatory signaling that can compromise erythropoiesis and lead to erythroid cell death. Meta-analysis from multiple studies showed the levels of pro-inflammatory cytokines, such as TNF-α, IL-6, and IL-8 were significantly higher in MDS patients as compared with controls (Shi et al., Medicine (Baltimore). 2019; 98: e15844). Moreover, the levels of these cytokines are higher in lower risk patients compared to higher risk MDS patients (Shetty et al., Leuk Res. 1996; 20: 891-900). NF-kB pathway is central in regulating these cytokines (Liu et al., Signal Transduct Target Ther. 2017; 2). Mesenchymal activation of NF-kB signaling is common in LR-MDS and activation of NF-κB drive inflammatory programs that attenuates hematopoiesis in low risk myelodysplastic syndromes (Ping et al., Leukemia. 2019; 33: 536-41). BET proteins regulate gene expression of key oncogenic pathways, such as NF-kB or TGF beta signaling, which are important drivers of proinflammatory signaling and ineffective hematopoiesis in MDS.

Pelabresib (CPI-0610) is a selective and potent small molecule BET inhibitor that has shown clinical activity in lymphomas and myelofibrosis (MF) in which NF-kB signaling and pro-inflammatory cytokine expression are relevant drivers of the disease process. In lymphoma and MF patients, pelabresib can decrease the levels of inflammatory cytokine regulated by the NF-κB pathway (Blum et al., Annals of Oncology. 2018; 29 and Talpaz et al., EHA Library. 2020; 293580).

In addition to improving hematopoiesis through reducing pro-inflammatory environment, pelabresib may also directly promote erythropoiesis. Among patients enrolled in the pelabresib clinical trial (Arm 1 of the MANIFEST trial (NCT2158858)), investigating pelabresib monotherapy in advanced MF patients refractory or intolerant to JAK inhibitors, 57.9% (11 out of 19) of them achieved ≥1.5 g/dL increase in Hgb levels without transfusion (Talpaz et al., EHA Library. 2020; 293580). Consistent with this, exploratory analysis of erythroid progenitors by immunohistochemistry staining for CD71 was conducted centrally on all available bone marrow biopsy pairs collected at baseline and Week 24 for 37 patients. Semi-quantitative analysis revealed an increase in erythrocyte progenitors in 59% (22 out of 37) of patients (Mertz et al., American Society of Hematology 2020). Ex vivo proliferation/differentiation studies showed that pelabresib promotes erythrocyte maturation, using CD34+ cells isolated from blood samples collected from healthy donors as well as MF patients and differentiated in the presence of a cytokine cocktail. Moreover, pelabresib treatment rescued the suppressive effects of ruxolitinib on erythroid differentiation in a dose-dependent manner (Mertz et al., American Society of Hematology 2020).

Further, in a phase 2 trial using pelabresib as monotherapy or in combination with ruxolitinib for patients with refractory advanced MF, treatment with pelabresib was able to induce erythroid responses. In Arm 1 of the study, transfusion dependent relapsed/refractory MF patients, 21% achieved RBC-TI≥12 weeks for a median duration of 44 weeks (Talpaz et al., EHA Library. 2020; 293580), In Arm 2 of the study, 36% of transfusion dependent MF patients who received pelabresib in addition to ruxolitinib achieved RBC-TI≥12 weeks for a median duration of 39 weeks (Verstovsek et al., Blood. 2020; 136: 51-52).

Provided herein, therefore are methods of using pelabresib, or a pharmaceutically acceptable salt thereof, for treating lower risk MDS, including low risk MDS and very low risk MDS.

Also provided herein are methods of using pelabresib, or a pharmaceutically acceptable salt thereof, for treating RBC transfusion dependent lower risk MDS, including RBC transfusion dependent low risk MDS and very low risk MDS.

Further provided herein are methods of using pelabresib, or a pharmaceutically acceptable salt thereof, for treating anemia associated with lower risk MDS, including low risk MDS and very low risk MDS.

Further provided herein are methods of using pelabresib, or a pharmaceutically acceptable salt thereof, for improving the hemoglobin level (e.g., increasing hemoglobin levels) in a subject suffering from lower risk MDS, including low risk MDS and very low risk MDS.

Further provided herein are methods of using pelabresib, or a pharmaceutically acceptable salt thereof, for treating cytopenia in a subject suffering from lower risk MDS, including low risk MDS and very low risk MDS.

Further provided herein are methods of using pelabresib, or a pharmaceutically acceptable salt thereof, for treating ineffective erythropoiesis in a subject suffering from lower risk MDS, including low risk MDS and very low risk MDS.

Further provided herein are methods of using pelabresib, or a pharmaceutically acceptable salt thereof, for improving bone marrow fibrosis in a subject suffering from lower risk MDS, including low risk MDS and very low risk MDS.

Further provided herein are methods of using pelabresib, or a pharmaceutically acceptable salt thereof, for normalizing platelets in a subject suffering from lower risk MDS, including low risk MDS and very low risk MDS.

Further provided herein are methods of using pelabresib, or a pharmaceutically acceptable salt thereof, for reducing spleen size in a subject suffering from lower risk MDS, including low risk MDS and very low risk MDS.

Further provided herein are methods of using pelabresib, or a pharmaceutically acceptable salt thereof, for reducing the transfusion burden in a subject suffering from lower risk MDS, including low risk MDS and very low risk MDS, and who is transfusion dependent prior to treatment.

### DETAILED DESCRIPTION

In a first embodiment, provided herein is a method for treating lower risk MDS, including low risk MDS and very low risk MDS, in a subject in need thereof, comprising administering to the subject an effective amount of pelabresib, or a pharmaceutically acceptable salt thereof. Alternatively, as part of a first embodiment, provided herein is the use of pelabresib, or a pharmaceutically acceptable salt thereof, for treating lower risk MDS, including low risk MDS and very low risk MDS. In another alternative, as part of a first embodiment, provided herein is the use of pelabresib, or a pharmaceutically acceptable salt thereof, for the manufacture of a medicament for treating lower risk MDS, including low risk MDS and very low risk MDS. In yet another alternative, as part of a first embodiment, provided herein is a pharmaceutical composition comprising pelabresib, or a pharmaceutically acceptable salt thereof, for treating lower risk MDS, including low risk MDS and very low risk MDS.

In a second embodiment, provided herein is a method for treating RBC transfusion dependent lower risk MDS, including RBC transfusion dependent low risk MDS and very low risk MDS, in a subject in need thereof, comprising administering to the subject an effective amount of pelabresib, or a pharmaceutically acceptable salt thereof. Alternatively, as part of a second embodiment, provided herein is the use of pelabresib, or a pharmaceutically acceptable salt thereof, for treating RBC transfusion dependent lower risk MDS, including RBC transfusion dependent low risk MDS and very low risk MDS. In another alternative, as part of a second embodiment, provided herein is the use of pelabresib, or a pharmaceutically acceptable salt thereof, for the manufacture of a medicament for treating RBC transfusion dependent lower risk MDS, including RBC transfusion dependent low risk MDS and very low risk MDS. In yet another alternative, as part of a second embodiment, provided herein is a pharmaceutical composition comprising pelabresib, or a pharmaceutically acceptable salt thereof, for treating RBC transfusion dependent lower risk MDS, including RBC transfusion dependent low risk MDS and very low risk MDS.

In a third embodiment, provided herein is a method for treating anemia associated with lower risk MDS, including low risk MDS and very low risk MDS, in a subject in need thereof, comprising administering to the subject an effective amount of pelabresib, or a pharmaceutically acceptable salt thereof. Alternatively, as part of a third embodiment, provided herein is the use of pelabresib, or a pharmaceutically acceptable salt thereof, for treating anemia associated with lower risk MDS, including low risk MDS and very low risk MDS. In another alternative, as part of a third embodiment, provided herein is the use of pelabresib, or a pharmaceutically acceptable salt thereof, for the manufacture of a medicament for treating anemia associated with lower risk MDS, including low risk MDS and very low risk MDS. In yet another alternative, as part of a third embodiment, provided herein is a pharmaceutical composition comprising pelabresib, or a pharmaceutically acceptable salt thereof, for treating anemia associated with lower risk MDS, including low risk MDS and very low risk MDS.

In a fourth embodiment, provided herein is a method for improving (e.g., increasing) the hemoglobin level in a subject suffering from lower risk MDS, including low risk MDS and very low risk MDS, comprising administering to the subject an effective amount of pelabresib, or a pharmaceutically acceptable salt thereof. Alternatively, as part of a fourth embodiment, provided herein is the use of pelabresib, or a pharmaceutically acceptable salt thereof, for improving (e.g., increasing) the hemoglobin level in a subject suffering from lower risk MDS, including low risk MDS and very low risk MDS. In another alternative, as part of a fourth embodiment, provided herein is the use of pelabresib, or a pharmaceutically acceptable salt thereof, for the manufacture of a medicament for improving (e.g., increasing) the hemoglobin level in a subject suffering from lower risk MDS, including low risk MDS and very low risk MDS. In yet another alternative, as part of a fourth embodiment, provided herein is a pharmaceutical composition comprising pelabresib, or a pharmaceutically acceptable salt thereof, for improving (e.g., increasing) the hemoglobin level in a subject suffering from lower risk MDS, including low risk MDS and very low risk MDS.

In a fifth embodiment, provided herein is a method for treating cytopenia in a subject suffering from lower risk MDS, including low risk MDS and very low risk MDS, comprising administering to the subject an effective amount of pelabresib, or a pharmaceutically acceptable salt thereof. Alternatively, as part of a fifth embodiment, provided herein is the use of pelabresib, or a pharmaceutically acceptable salt thereof, for treating cytopenia in a subject suffering from lower risk MDS, including low risk MDS and very low risk MDS. In another alternative, as part of a fifth embodiment, provided herein is the use of pelabresib, or a pharmaceutically acceptable salt thereof, for the manufacture of a medicament for treating cytopenia in a subject suffering from lower risk MDS, including low risk MDS and very low risk MDS. In yet another alternative, as part of a fifth embodiment, provided herein is a pharmaceutical composition comprising pelabresib, or a pharmaceutically acceptable salt thereof, for treating cytopenia in a subject suffering from lower risk MDS, including low risk MDS and very low risk MDS.

In a sixth embodiment, provided herein is a method for treating ineffective erythropoiesis in a subject suffering from lower risk MDS, including low risk MDS and very low risk MDS, comprising administering to the subject an effective amount of pelabresib, or a pharmaceutically acceptable salt thereof. Alternatively, as part of a sixth embodiment, provided herein is the use of pelabresib, or a pharmaceutically acceptable salt thereof, for treating ineffective erythropoiesis in a subject suffering from lower risk MDS, including low risk MDS and very low risk MDS. In another alternative, as part of a sixth embodiment, provided herein is the use of pelabresib, or a pharmaceutically acceptable salt thereof, for the manufacture of a medicament for treating ineffective erythropoiesis in a subject suffering from lower risk MDS, including low risk MDS and very low risk MDS. In yet another alternative, as part of a sixth embodiment, provided herein is a pharmaceutical composition comprising pelabresib, or a pharmaceutically acceptable salt thereof, for treating ineffective erythropoiesis in a subject suffering from lower risk MDS, including low risk MDS and very low risk MDS.

In a seventh embodiment, provided herein is a method for improving bone marrow fibrosis in a subject suffering from lower risk MDS, including low risk MDS and very low risk MDS, comprising administering to the subject an effective amount of pelabresib, or a pharmaceutically acceptable salt thereof. Alternatively, as part of a seventh embodiment, provided herein is the use of pelabresib, or a pharmaceutically acceptable salt thereof, for improving bone marrow fibrosis in a subject suffering from lower risk MDS, including low risk MDS and very low risk MDS. In another alternative, as part of a seventh embodiment, provided herein is the use of pelabresib, or a pharmaceutically acceptable salt thereof, for the manufacture of a medicament for improving bone marrow fibrosis in a subject suffering from lower risk MDS, including low risk MDS and very low risk MDS. In yet another alternative, as part of a seventh embodiment, provided herein is a pharmaceutical composition comprising pelabresib, or a pharmaceutically acceptable salt thereof, for improving bone marrow fibrosis in a subject suffering from lower risk MDS, including low risk MDS and very low risk MDS.

In an eighth embodiment, provided herein is a method for normalizing platelets in a subject suffering from lower risk MDS, including low risk MDS and very low risk MDS, comprising administering to the subject an effective amount of pelabresib, or a pharmaceutically acceptable salt thereof. Alternatively, as part of an eighth embodiment, provided herein is the use of pelabresib, or a pharmaceutically acceptable salt thereof, for normalizing platelets in a subject suffering from lower risk MDS, including low risk MDS and very low risk MDS. In another alternative, as part of an eighth embodiment, provided herein is the use of pelabresib, or a pharmaceutically acceptable salt thereof, for the manufacture of a medicament for normalizing platelets in a subject suffering from lower risk MDS, including low risk MDS and very low risk MDS. In yet another alternative, as part of an eighth embodiment, provided herein is a pharmaceutical composition comprising pelabresib, or a pharmaceutically acceptable salt thereof, for normalizing platelets in a subject suffering from lower risk MDS, including low risk MDS and very low risk MDS.

In a ninth embodiment, provided herein is a method for reducing spleen size in a subject suffering from lower risk MDS, including low risk MDS and very low risk MDS, comprising administering to the subject an effective amount of pelabresib, or a pharmaceutically acceptable salt thereof. Alternatively, as part of a ninth embodiment, provided herein is the use of pelabresib, or a pharmaceutically acceptable salt, for reducing spleen size in a subject suffering from lower risk MDS, including low risk MDS and very low risk MDS. In another alternative, as part of a ninth embodiment, provided herein is the use of pelabresib, or a pharmaceutically acceptable salt thereof, for the manufacture of a medicament for reducing spleen size in a subject suffering from lower risk MDS, including low risk MDS and very low risk MDS. In yet another alternative, as part of a ninth embodiment, provided herein is a pharmaceutical composition comprising pelabresib, or a pharmaceutically acceptable salt thereof, for reducing spleen size in a subject suffering from lower risk MDS, including low risk MDS and very low risk MDS.

In a tenth embodiment, provided herein is a method for reducing the transfusion burden in a subject suffering from lower risk MDS, including low risk MDS and very low risk MDS, and who is transfusion dependent prior to treatment, comprising administering to the subject an effective amount of pelabresib, or a pharmaceutically acceptable salt thereof. Alternatively, as part of a tenth embodiment, provided herein is the use of pelabresib, or a pharmaceutically acceptable salt, for reducing the transfusion burden in a subject suffering from lower risk MDS, including low risk MDS and very low risk MDS, and who is transfusion dependent prior to treatment. In another alternative, as part of a tenth embodiment, provided herein is the use of pelabresib, or a pharmaceutically acceptable salt thereof, for the manufacture of a medicament for reducing the transfusion burden in a subject suffering from lower risk MDS, including low risk MDS and very low risk MDS, and who is transfusion dependent prior to treatment. In yet another alternative, as part of a tenth embodiment, provided herein is a pharmaceutical composition comprising pelabresib, or a pharmaceutically acceptable salt thereof, for reducing the transfusion burden in a subject suffering from lower risk MDS, including low risk MDS and very low risk MDS, and who is transfusion dependent prior to treatment.

Pelabresib (CPI-0610), or 2-((4S)-6-(4-chlorophenyl)-1-methyl-4H-benzo[c]isoxazolo[4,5-e]azepin-4-yl)acetamide, is exemplified as Compound 144 in U.S. Pat. No. 8,796,261, and has the following structural formula:

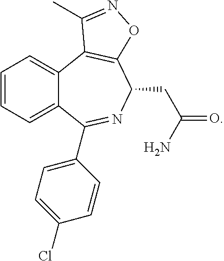 [SMILES: Cc1noc2c1-c1ccccc1C(c1ccc(Cl)cc1)=N[C@H]2CC(N)=O]

The term pelabresib as used herein includes crystalline and/or hydrated forms of pelabresib such as the monohydrate and crystalline Form A monohydrate that are disclosed in U.S. Pat. No. 9,969,747, and, in one aspect, are included as part of the invention.

Pelabresib or the pharmaceutically acceptable salts described herein can be formulated as pharmaceutical compositions and administered to a subject, such as a human, in a variety of forms adapted to the chosen route of administration. Typical routes of administering such pharmaceutical compositions include, without limitation, oral, topical, buccal, transdermal, inhalation, parenteral, sublingual, rectal, vaginal, and intranasal. The term parenteral as used herein includes subcutaneous injections, intravenous, intramuscular, intrathecal, intrasternal injection or infusion techniques. Methods of formulating pharmaceutical compositions are well known in the art, for example, as disclosed in “Remington: The Science and Practice of Pharmacy,” University of the Sciences in Philadelphia, ed., 21st edition, 2005, Lippincott, Williams & Wilkins, Philadelphia, PA.

As used herein, the term “lower risk MDS” refers to MDS that is characterized as being low risk or very low risk according to the IPSS-R Prognostic Risk Categories/Scores. See Greenberg P. L., et al. Revised international prognostic scoring system for myelodysplastic syndromes. Blood. 2012; 120: 2454-65.

As used herein, the term “low risk MDS” refers to low risk MDS as defined by IPSS-R Prognostic Risk Categories/Scores. See Greenberg P. L., et al. Revised international prognostic scoring system for myelodysplastic syndromes. Blood. 2012; 120: 2454-65. For example, low risk MDS is MDS in which the subject has an IPSS-R Prognostic Risk score of >1.5-3.

As used herein, the term “very low risk MDS” refers to very low risk MDS as defined by IPSS-R Prognostic Risk Categories/Scores. See Greenberg P. L., et al. Revised international prognostic scoring system for myelodysplastic syndromes. Blood. 2012; 120: 2454-65. For example, very low risk MDS is MDS in which the subject has an IPSS-R Prognostic Risk score of ≤1.5.

The term “lower risk MDS-associated anemia”, “anemia associated with lower risk MDS”, and “anemia due to lower risk MDS” are synonymous and refer to anemia that has developed or has been acquired in a subject as a result of having or suffering from lower risk MDS.

The terms “subject” and “patient” are synonymous and refer to a mammal in need of treatment, e.g., companion animals (e.g., dogs, cats, and the like), farm animals (e.g., cows, pigs, horses, sheep, goats, and the like) and laboratory animals (e.g., rats, mice, guinea pigs, and the like). Unless stated to the contrary, the subject is a human in need of treatment.

The terms “administer,” “administering,” or “administration” refer to providing, implanting, absorbing, ingesting, injecting, inhaling, or otherwise introducing pelabresib, or a pharmaceutically acceptable salt or composition thereof, to, in or on a subject.

The terms “treatment,” “treat,” and “treating” refer to reversing, alleviating, delaying the onset of, or inhibiting the progress of a disease or one or more symptoms of a disease described herein. In some embodiments, treatment may be administered after one or more signs or symptoms of the disease have developed or have been observed (i.e., therapeutic treatment). In other embodiments, treatment may be administered in the absence of signs or symptoms of the disease. For example, treatment may be administered to a susceptible subject prior to the onset of symptoms (i.e., prophylactic treatment). Treatment may also be continued after symptoms have resolved, for example, to delay or prevent recurrence. In certain embodiments, treatment includes delaying onset of at least one symptom of the disorder for a period of time.

The terms an “effective amount” or “therapeutically effective amount” of pelabresib or a pharmaceutically acceptable salt thereof described herein refer to an amount of a pelabresib or a pharmaceutically acceptable salt thereof that is sufficient to provide a therapeutic benefit in the treatment of a condition described herein. In one aspect, an effective amount is between about 0.01 to about 100 mg/kg body weight/day of pelabresib or a pharmaceutically acceptable salt thereof, such as, e.g., about 0.1 to about 100 mg/kg body weight/day.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. In one aspect, pelabresib, or a pharmaceutically acceptable salt thereof, may be formulated at a dose of from about 50 mg to about 500 mg for e.g., administration once, twice, or three times daily. In another aspect, 2-((4S)-6-(4-chlorophenyl)-1-methyl-4H-benzo[c]isoxazolo[4,5-e]azepin-4-yl)acetamide or a pharmaceutically acceptable salt thereof, may be formulated at a dose of from about 50 mg to about 500 mg for e.g., administration once, twice, or three times daily. In another aspect, 2-((4S)-6-(4-chlorophenyl)-1-methyl-4H-benzo[c]isoxazolo[4,5-e]azepin-4-yl)acetamide or a pharmaceutically acceptable salt thereof, may be formulated at a dose of from about 50 mg to about 500 mg for e.g., administration once, twice, or three times daily, wherein 2-((4S)-6-(4-chlorophenyl)-1-methyl-4H-benzo[c]isoxazolo[4,5-e]azepin-4-yl)acetamide is a monohydrate or crystalline Form A monohydrate. For example, pelabresib may be administered at a dosage of from about 50 mg to about 300 mg/day, from about 50 mg to about 175 mg/day, from about 50 mg to about 150 mg/day, from about 75 mg to about 300 mg/day, from about 75 mg to about 200 mg/day, from about 75 mg to about 175 mg/day, from about 75 mg to about 150 mg/day, from about 70 mg to about 160 mg/day, from about 100 mg to about 300 mg/day, from about 150 mg to about 250 mg/day, or at about 50 mg/day, at about 75 mg/day, or at about 125 mg/day, about 150 mg/day, about 175 mg/day, about 200 mg/day, about 225 mg/day, or about 250 mg/day. In one aspect, pelabresib monohydrate, may be formulated at a dose of from about 50 mg to about 500 mg for e.g., administration once, twice, or three times daily. For example, pelabresib monohydrate may be administered at a dosage of from about 50 mg to about 300 mg/day, from about 50 mg to about 175 mg/day, from about 50 mg to about 150 mg/day, from about 75 mg to about 300 mg/day, from about 75 mg to about 175 mg/day, from about 75 mg to about 150 mg/day, from about 70 mg to about 160 mg/day, from 100 about mg to about 300 mg/day, from about 150 mg to about 250 mg/day, or at about 50 mg/day, at about 75 mg/day, at 1 about 25 mg/day, about 150 mg/day, about 175 mg/day, about 200 mg/day, about 225 mg/day, or about 250 mg/day. In one aspect, monohydrate crystalline Form A of pelabresib as described herein, may be formulated at a dose of from about 50 mg to about 500 mg for e.g., administration once, twice, or three times daily. For example, monohydrate crystalline Form A of pelabresib as described herein may be administered at a dosage of from about 50 mg to about 300 mg/day, from about 50 mg to about 175 mg/day, from about 50 mg to about 150 mg/day, from about 75 mg to about 300 mg/day, from about 75 mg to about 175 mg/day, from about 75 mg to about 150 mg/day, from about 70 mg to about 160 mg/day, from 100 about mg to about 300 mg/day, from about 150 mg to about 250 mg/day, or at about 50 mg/day, at about 75 mg/day, at 1 about 25 mg/day, about 150 mg/day, about 175 mg/day, about 200 mg/day, about 225 mg/day, or about 250 mg/day. In one aspect, pelabresib, or a pharmaceutically acceptable salt thereof, may be formulated at a dose of from 50 mg to 500 mg for e.g., administration once, twice, or three times daily. For example, pelabresib may be administered at a dosage of from 50 mg to 300 mg/day, from 50 mg to 175 mg/day, from 50 mg to 150 mg/day, from 75 mg to 300 mg/day, from 75 mg to 175 mg/day, from 75 mg to 150 mg/day, from 70 mg to 160 mg/day, from 100 mg to 300 mg/day, from 150 mg to 250 mg/day, or at 50 mg/day, 75 mg/day, 125 mg/day, 150 mg/day, 175 mg/day, 200 mg/day, 225 mg/day, or 250 mg/day. In one aspect, pelabresib, Form A monohydrate may be formulated at a dose of from 50 mg to 500 mg for e.g., administration once, twice, or three times daily. For example, pelabresib Form A monohydrate may be administered at a dosage of from 50 mg to 300 mg/day, from 50 mg to 175 mg/day, from 50 mg to 150 mg/day, from 75 mg to 300 mg/day, from 75 mg to 200 mg/day, from 75 mg to 175 mg/day, from 75 mg to 150 mg/day, from 70 mg to 160 mg/day, from 100 mg to 300 mg/day, from 150 mg to 250 mg/day, or at 50 mg/day, 75 mg/day, 125 mg/day, 150 mg/day, 175 mg/day, 200 mg/day, 225 mg/day, or 250 mg/day. In one aspect, monohydrate crystalline Form A of pelabresib as described herein may be formulated at a dose of from 50 mg to 500 mg for e.g., administration once, twice, or three times daily. For example, monohydrate crystalline Form A of pelabresib as described herein may be administered at a dosage of from 50 mg to 300 mg/day, from 50 mg to 175 mg/day, from 50 mg to 150 mg/day, from 75 mg to 300 mg/day, from 75 mg to 200 mg/day, from 75 mg to 175 mg/day, from 75 mg to 150 mg/day, from 70 mg to 160 mg/day, from 100 mg to 300 mg/day, from 150 mg to 250 mg/day, or at 50 mg/day, 75 mg/day, 125 mg/day, 150 mg/day, 175 mg/day, 200 mg/day, 225 mg/day, or 250 mg/day.

As used herein, the recitation of a range of values is intended to serve as a shorthand method of referring individually to each separate value falling within the range as well as the highest and lowest values that define the range and each value is incorporated into the specification as if it were individually recited herein, unless expressly stated to the contrary. For example, a range of values from X to Y includes both X and Y and all the values in between X and Y.

The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

In an eleventh embodiment, the pelabresib used in the described methods (e.g., as in any one of the first to tenth embodiments), is crystalline. Alternatively, as part of an eleventh embodiment, the pelabresib used in the described methods is a hydrate. In another alternative, as part of an eleventh embodiment, the pelabresib used in the described methods is a monohydrate. Alternatively, as part of an eleventh embodiment, the pelabresib used in the described methods (e.g., as in any one of the first to tenth embodiments), is crystalline Form A (e.g., monohydrate crystalline Form A) characterized by at least three, at least four, at least five, or by six x-ray powder diffraction peaks at 2Θ angles selected from 4.73°, 18.09°, 18.48°, 18.80°, 19.70°, and 25.17°. In another alternative, as part of an eleventh embodiment, the pelabresib used in the described methods (e.g., as in any one of the first to tenth embodiments), is crystalline Form A (e.g., monohydrate crystalline Form A) characterized by x-ray powder diffraction peaks at 2Θ angles 4.73°, 9.42°, 12.91°, 18.09°, 18.48°, 18.80°, 19.70°, 21.42°, and 25.17°. In yet another alternative, as part of an eleventh embodiment, the pelabresib used in the described methods (e.g., as in any one of the first to tenth embodiments), is crystalline Form A (e.g., monohydrate crystalline Form A) characterized x-ray powder diffraction peaks at 2Θ angles 4.73°, 8.11°, 9.42°, 12.91°, 14.10°, 14.97°, 18.09°, 18.48°, 18.80°, 19.70°, 21.42°, and 25.17°, 26.07°, and 26.53°. Additional details on the characterization of crystalline Form A (e.g., monohydrate crystalline Form A) as well as hydrated forms of pelabresib is found in U.S. Pat. No. 9,969,747, the contents of which are incorporated herein by reference.

It will be understood that the 2-theta values of the X-ray powder diffraction patterns for monohydrate crystalline Form A may vary slightly from one instrument to another and also depending on variations in sample preparation and batch to batch variation. For example, without wishing to be bound by theory, it is believed that some variations in 2-theta values are attributable to the amount of water comprised in the crystalline lattice, e.g., in the case of hydrated (such as a monohydrate) and anhydrous forms. Therefore, the XRPD patterns/assignments for crystalline Form A (e.g., monohydrate crystalline Form A) are not to be construed as absolute and can vary ±0.2 degrees, except for the 2Θ angles 8.11°, 14.10°, 14.97°, 26.07°, and 26.53° of crystalline Form A (e.g., monohydrate crystalline Form A), which can vary by ±0.3 degrees.

In a twelfth embodiment, a subject being treated by pelabresib, or pharmaceutically acceptable salt or composition thereof (including any one of the first through eleventh embodiments) is classified as a subject who is transfusion dependent (TD). Alternatively, as part of a twelfth embodiment, a subject being treated by pelabresib, or pharmaceutically acceptable salt or composition thereof (including any one of the first through eleventh embodiments) is classified as a subject who is transfusion dependent (TD) prior to treatment. As used herein, the term transfusion dependent (TD) refers to those subjects who require regular blood transfusions. The term “red blood cell (RBC) transfusion dependent LR-MDS” refers to LR-MDS in which the patient is also transfusion dependent (TD).

In a thirteenth embodiment, a subject being treated by pelabresib, or pharmaceutically acceptable salt or composition thereof (including any one of the first through twelfth embodiments) becomes transfusion independent during treatment. In some aspects, as part of a thirteenth embodiment, a subject being treated by pelabresib or pharmaceutically acceptable salt or composition thereof (including any one of the first through twelfth embodiments) becomes transfusion independent for a period of time during treatment. As used herein, the term transfusion independent (TI) refers to those subjects who do not require regular blood transfusions. In one aspect, as part of a thirteenth embodiment, a subject being treated by pelabresib or pharmaceutically acceptable salt or composition thereof (including any one of the first through eleventh embodiments) becomes transfusion independent, wherein the transfusion independence is characterized by the absence of a RBC transfusion for a period of at least about 4 consecutive weeks, at least about 6 consecutive weeks, at least about consecutive 8 weeks, at least about 10 consecutive weeks, or at least about 12 consecutive weeks during treatment. In one aspect, as part of a thirteenth embodiment, a subject being treated by pelabresib or pharmaceutically acceptable salt or composition thereof (including any one of the first through eleventh embodiments) becomes transfusion independent, wherein the transfusion independence is characterized by the absence of a RBC transfusion for a period of about 8 consecutive weeks during treatment. In one aspect, as part of a thirteenth embodiment, a subject being treated by pelabresib or pharmaceutically acceptable salt or composition thereof (including any one of the first through eleventh embodiments) becomes transfusion independent, wherein the transfusion independence is characterized by the absence of a RBC transfusion for a period of at least 10 consecutive days, at least about 15 consecutive days, at least about 20 consecutive days, at least about 25 consecutive days, at least about 30 consecutive days, at least about 35 consecutive days, at least about 40 consecutive days, at least about 45 consecutive days, at least about 50 consecutive days, at least about 55 consecutive days, at least about 60 consecutive days, at least about 70 consecutive days, at least about 75 consecutive days, at least about 80 consecutive days, or at least about 85 consecutive days during treatment. In one aspect, as part of a thirteenth embodiment, a subject being treated by pelabresib or pharmaceutically acceptable salt or composition thereof (including any one of the first through eleventh embodiments) becomes transfusion independent, wherein the transfusion independence is characterized by the absence of a RBC transfusion for a period of about 10 to about 90 consecutive days, about 20 to about 90 consecutive days, about 30 to about 90 consecutive days, about 40 to about 90 consecutive days, about 50 to about 90 consecutive days, about 50 to about 60 consecutive days, about 55 to about 58 consecutive days, about 80 to about 90 consecutive days, or about 82 to about 56 consecutive days during treatment. In one aspect, as part of a thirteenth embodiment, a subject being treated by pelabresib or pharmaceutically acceptable salt or composition thereof (including any one of the first through eleventh embodiments) becomes transfusion independent, wherein the transfusion independence is characterized by the absence of a RBC transfusion during any consecutive 56-day period after the start of treatment. In one aspect, as part of a thirteenth embodiment, a subject being treated by pelabresib or pharmaceutically acceptable salt or composition thereof (including any one of the first through eleventh embodiments) becomes transfusion independent, wherein the transfusion independence is characterized by the absence of a RBC transfusion during any consecutive 84-day period after the start of treatment.

In a fourteenth embodiment, a subject being treated by pelabresib or pharmaceutically acceptable salt or composition thereof (including any one of the first through thirteenth embodiments) experiences an improvement (e.g., increase) in hemoglobin levels following treatment. In one aspect, as part of a fourteenth embodiment, a subject being treated by pelabresib or pharmaceutically acceptable salt or composition thereof (including any one of the first through thirteenth embodiments) experiences an improvement in hemoglobin levels characterized as a mean hemoglobin increase of ≥0.5 g/dL, ≥1.0 g/dL, or ≥1.5 g/dL during treatment. In one aspect, as part of a fourteenth embodiment, a subject being treated by pelabresib or pharmaceutically acceptable salt or composition thereof (including any one of the first through thirteenth embodiments) experiences an improvement in hemoglobin levels characterized as a mean hemoglobin increase of ≥0.5 g/dL, ≥1.0 g/dL, or ≥1.5 g/dL during a period of at least about 4 consecutive weeks, at least about 6 consecutive weeks, at least about 8 consecutive weeks, at least about 10 consecutive weeks, or at least about 12 consecutive weeks during treatment, or for a period of time as otherwise described above in the twelfth embodiment, in which the subject is also transfusion independent. In one aspect, as part of a fourteenth embodiment, a subject being treated by pelabresib or pharmaceutically acceptable salt or composition thereof (including any one of the first through thirteenth embodiments) experiences as an improvement in hemoglobin levels characterized as a mean hemoglobin increase of ≥1.0 g/dL during a period of at least about 8 consecutive weeks in which the subject is also transfusion independent.

In a fifteenth embodiment, a subject being treated by pelabresib or pharmaceutically acceptable salt or composition thereof (including any one of the first through fourteenth embodiments) experiences as Erythroid Response (mHI-E). In one aspect, as part of a fifteenth embodiment, a subject being treated by pelabresib or pharmaceutically acceptable salt or composition thereof (including any one of the first through fourteenth embodiments) experiences as Erythroid Response (mHI-E) defined as an RBC transfusion reduction of ≥2, ≥3, ≥4, ≥5, or ≥6 units during treatment. In one aspect, as part of a fifteenth embodiment, a subject being treated by pelabresib or pharmaceutically acceptable salt or composition thereof (including any one of the first through fourteenth embodiments) experiences as Erythroid Response (mHI-E) defined as an RBC transfusion reduction of ≥2, ≥3, ≥4, ≥5, or ≥6 units during a period of at least at least about 4 consecutive weeks, at least about 6 consecutive weeks, at least about 8 consecutive weeks, at least about 10 consecutive weeks, or at least about 12 consecutive weeks during treatment, or for a period of time as otherwise described above in the thirteenth embodiment, in which the subject is also transfusion independent (for patients with a baseline RBC transfusion burden of ≥4 units/8 weeks). In one aspect, as part of a fifteenth embodiment, a subject being treated by pelabresib or pharmaceutically acceptable salt or composition thereof (including any one of the first through fourteenth embodiments) experiences as Erythroid Response (mHI-E) defined as an RBC transfusion reduction of ≥4 units during a period of at least about 8 consecutive weeks in which the subject is also transfusion independent (for patients with a baseline RBC transfusion burden of ≥4 units/8 weeks). In one aspect, as part of a fifteenth embodiment, a subject being treated by pelabresib or pharmaceutically acceptable salt or composition thereof (including any one of the first through fourteenth embodiments) experiences as Erythroid Response (mHI-E) defined as a mean hemoglobin increase of ≥0.5 g/dL, ≥1.0 g/dL, ≥1.5 g/dL, or ≥2.0 g/dL during a period of at least about 4 consecutive weeks, at least about 6 consecutive weeks, at least about 8 consecutive weeks, at least about 10 consecutive weeks, or at least about 12 consecutive weeks during treatment, or for a period of time as otherwise described above in the thirteenth embodiment, in which the subject is also transfusion independent (for patients with a baseline RBC transfusion burden of <4 units/8 weeks). In one aspect, as part of a fifteenth embodiment, a subject being treated by pelabresib or pharmaceutically acceptable salt or composition thereof (including any one of the first through fourteenth embodiments) experiences as Erythroid Response (mHI-E) defined as a mean hemoglobin increase of ≥1.5 g/dL during a period of at least about 8 consecutive weeks, in which the subject is also transfusion independent (for patients with a baseline RBC transfusion burden of <4 units/8 weeks).

In a sixteenth embodiment, a subject being treated by pelabresib or pharmaceutically acceptable salt or composition thereof (including any one of the first through fifteenth embodiments) experiences a Neutrophil Response (HI-N). In one aspect, as part of a sixteenth embodiment, a subject being treated by pelabresib or pharmaceutically acceptable salt or composition thereof (including any one of the first through fifteenth embodiments) experiences a Neutrophil Response (HI-N) defined as a relative increase of ≥100% and an absolute increase of >0.5×10⁹/L from baseline in neutrophil count at every assessment during any consecutive 8-week period post baseline, for patients with baseline neutrophil count of <1.0×10⁹/L.

In a seventeenth embodiment, a subject being treated by pelabresib or pharmaceutically acceptable salt or composition thereof (including any one of the first through sixteenth embodiments) has a platelet count of ≥75×10⁹/L prior to treatment. Alternatively, as part of a seventeenth embodiment, a subject being treated by pelabresib or pharmaceutically acceptable salt or composition thereof (including any one of the first through sixteenth embodiments) has a platelet count of <75×10⁹/L prior to treatment.

In an eighteenth embodiment, a subject being treated by pelabresib or pharmaceutically acceptable salt or composition thereof (including any one of the first through seventeenth embodiments) experiences a Platelet Response (HI-P) during treatment. In one aspect, as part of an eighteenth embodiment, a subject being treated by pelabresib or pharmaceutically acceptable salt or composition thereof (including any one of the first through seventeenth embodiments) experiences a Platelet Response (HI-P) defined by an absolute increase of ≥30×10⁹/L from baseline in platelet count (for patients with a baseline platelet count between 20×10⁹/L and 100×10⁹/L), or a platelet count of >20×10⁹/L and a relative increase of ≥100% from baseline (for patients with a baseline platelet count of <20×10⁹/L), at every assessment during any consecutive 8-week period post baseline.

In a nineteenth embodiment, a subject being treated by the methods described herein (including any one of the first through eighteenth embodiments) is administered pelabresib (e.g., pelabresib monohydrate or pelabresib monohydrate crystalline Form A) at a dose ranging from about 50 mg/day to about 200 mg/day. Alternatively, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose ranging from about 100 mg/day to about 150 mg/day. In another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose ranging from about 150 mg/day to about 200 mg/day. In yet another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose ranging from about 50 mg/day to about 100 mg/day. In yet another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose of about 75 mg/day, about 100 mg/day, about 125 mg/day, about 150 mg/day, or about 175 mg/day. In yet another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose of about 75 mg/day. In yet another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose of about 125 mg/day. In yet another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose of about 175 mg/day. In yet another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose of about 125 mg/day, provided the subject has a baseline platelet count of ≥75×10⁹/L prior to treatment. In yet another alternative, as part of a nineteenth embodiment, a subject being treated by the methods described herein (including any one of the first through eighteenth embodiments) is administered pelabresib (e.g., pelabresib monohydrate or pelabresib monohydrate crystalline Form A) at a dose of about 75 mg/day, provided the subject has a baseline platelet count of <75×10⁹/L prior to treatment. In another alternative, as part of a nineteenth embodiment, a subject being treated by the methods described herein (including any one of the first through eighteenth embodiments).

### EXEMPLIFICATION

### Investigating the Impact of Pelabresib on Cytokine Secretion of Primary MDS Patient Cells

This study was performed to evaluate the anti-inflammatory activity of pelabresib on MDS patient cells from different donors. The NF-kB pathway is central in regulating cytokines such as TNF-α, IL-6, and IL-8 which are elevated in MDS patients and play role in the pathogenesis of myelodysplastic syndromes (Ref: e.g. doi: 10.1097/MD.0000000000015844).

Primary MDS patient samples (cryopreserved peripheral blood mononuclear cells (PBMCs)) were purchased from Discovery Life Sciences. For sample selection, we chose patients with low blast count (ideally below 5%), excluded patients with multilineage dysplasia and (if available) checked blood parameters such as white blood cell count and disease staging notes in order to select patients that show LR-MDS characteristics. Freshly thawed PBMCs were seeded at 50 000 cells/well in a 96-well flat bottom plate. 0.001 μg/mL lipopolysaccharide (LPS-B5, Invivogen) were added to induce IL-6 production. In addition, a nine-point dose titration series of pelabresib (CPI-0610) with concentrations ranging from 5 μM to 3.3 nM in duplicate was added. The absolute amount of DMSO in each well was kept constant. “DMSO only” served as control for maximum IL-6 secretion. The assay plates were incubated for approximately 18 hours 37° C., 5% CO₂. After centrifugation, the supernatant of the cells was collected and IL-6 protein in the supernatant was quantified using Human IL-6 Kit (V-PLEX, Meso Scale Discovery). The assay plates were read on a Meso Sector S600. Data analysis: In Microsoft Excel the average background signal (0 pg/mL of calibrator brand served as background control) was subtracted from the raw data of all wells. Values of compound-treated wells were normalized to “DMSO only” control, which was set to 100% IL-6 release. The averages of the normalized values of each experiment were plotted using GraphPad Prism 8. The half maximal inhibitory concentration (IC50) was calculated by logarithmic transformation of the compound concentrations and applying “log(inhibitor) vs. response—variable slope (four parameters)”—function. Mean t SD and fitted curves were displayed in the graphs.

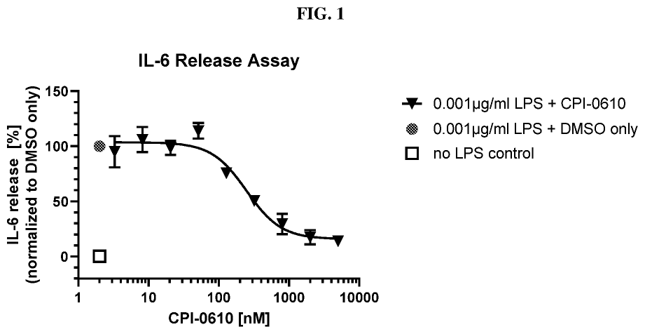

As shown by FIG. 1, pelabresib suppressed IL-6 release in donor PBMCs in a dose dependent manner. Values were normalized to “DMSO only” control, n=2 donors, mean±SD, IC50=248 nM.

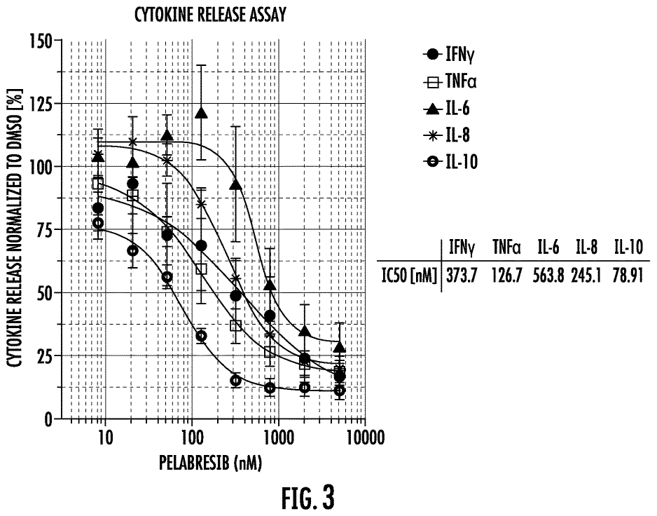

Additional studies were performed using an eight-point dose titration series of pelabresib (CPI-0610) with concentrations ranging from 5 μM to 8.2 nM in triplicate, following the same procedure outlined above. After centrifugation, the supernatant of the cells was collected and IFNγ, TNFα, IL-6, IL-8 or IL-10 protein in the supernatant was quantified using Proinflammatory Panel 1 Human Kit (IFNγ, TNFα, IL-6 and IL-10) or Human IL-8 Kit (both V-PLEX, Meso Scale Discovery). The assay plates were read on a Meso Sector S600. Data analysis: In Microsoft Excel the average background signal (0 pg/mL of calibrator brand served as background control) was subtracted from the raw data of all wells. Values of compound-treated wells were normalized to “DMSO only” control, which was set to 100% cytokine release. The averages of the normalized values of all experiments per cytokine were plotted using GraphPad Prism 8. The half maximal inhibitory concentration (IC₅₀) was calculated by logarithmic transformation of the compound concentrations and applying “log(inhibitor) vs. response −variable slope (four parameters)”—function. Mean±SEM and fitted curves were displayed in the graphs. Results are shown in FIG. 3, where pelabresib suppressed release of IFNγ, TNFα, IL-6, IL-8, and IL-10.

Investigating the Effect of Pelabresib on the Erythropoiesis of Hematopoietic Stem Cells Isolated from MDS Patient Bone Marrow

Primary MDS patient samples (cryopreserved bone marrow mononuclear cells (BMMCs)) were purchased from Discovery Life Sciences. For sample selection, we chose patients with low blast count (ideally below 5%), excluded patients with multilineage dysplasia and (if available) checked blood parameters such as white blood cell count and disease staging notes in order to select patients that show LR-MDS characteristics.

Hematopoietic stem cells (CD34+) were isolated with EasySep Human CD34+ selection Kit II (StemCell Technologies) using freshly thawed cells. Cells were seeded at 1000 cells/well (for erythropoiesis) or 2500 cells/well (for CC100 differentiation mix) in a 96-flat-well plate. Differentiation cocktails StemSpan™ Erythroid Expansion Supplement (for Erythropoieses) or StemSpan™ CC100 (differentiation mix) were added according to the manufacturer's instructions. A 5-point titration series of pelabresib monohydrate with concentrations ranging from 500 nM to 31.25 nM was tested in duplicate. “DMSO only” treated wells served as control and were also used to prepare flow cytometry controls (such as FMOs). On day three or four after seeding a feeding step was performed for erythropoiesis. On day 7 cells were analysed via flow cytometry. The cell staining was performed according to the manufacturer's instructions using the following antibodies: BV421 Anti-Human CD34 [clone:561] (BioLegend), AF700 Anti-Human CD71 [clone: M-A712] (BD Biosciences), FITC Anti-Human CD235a [clone: GA-R2 (HIR2)] (BD Biosciences) and Dye eFluor506 Invitrogen (Live/Dead) (Invitrogen/ThermoFisher) to discriminate live from dead cells. Cells were acquired on a BD FACS LSR×20 Fortessa. Gating was performed as follows: exclusion of debris→single cells→viable cells. Among the viable cells the percentage of CD34+CD71+ (megakaryocyte-erythroid progenitors (MEP)) and percentage of CD34−CD71+ (pro-erythroblasts/erythroblasts (pro-EB/EB)) was determined and depicted in GraphPad Prism 8 per donor.

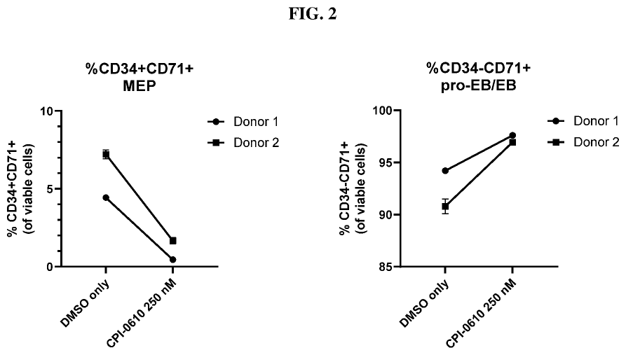

As shown by FIG. 2, pelabresib enhanced the differentiation of CD34+ stem cells derived from LR-MDS donors towards erythropoiesis.

Phase 2 Study of Pelabresib in Transfusion-Dependent Patients with Lower Risk Myelodysplastic Syndrome (MDS)

A Phase 2b study evaluating the efficacy and safety of pelabresib in transfusion-dependent patients with lower-risk MDS will be conducted.

### Study Design

This Phase 2 part of the study is a multicenter open-label study of pelabresib treatment in patients with lower risk MDS. Pelabresib monohydrate will be orally administered once daily (QD) for 14 consecutive days followed by a 7-day break, which is considered 1 cycle of treatment (1 cycle=21 days), until unacceptable toxicity, disease progression or other discontinuation criteria (i.e., need of intervention/therapy precluded by eligibility criteria, patient noncompliance, pregnancy, or other medical condition that interferes with study participation).

Patients with a baseline platelet count ≥75×10⁹/L will receive a starting dose of 125 mg QD or 150 mg QD of pelabresib monohydrate. Patients with a baseline platelet count <75×10⁹/L will start at a dose of 75 mg QD or patient with a baseline platelet count of ≥50×10⁹/L<75×10⁹/L will start at a dose of 75 mg QD. The pelabresib monohydrate dose may be increased in increments of 25 mg QD, no more often than once every 2 cycles, up to a maximum of 175 mg QD or 200 mg QD, provided 1) platelet count is ≥75×10⁹/L, 2) absolute neutrophil count (ANC) is ≥750×10⁶/L in the absence of growth factors, 3) no bleeding incident(s) are experienced, and 4) no ≥Grade 3 adverse event(s) (AEs) attributed to pelabresib are observed. Clinical evaluations will be performed in patients with baseline PLT counts ≥50×10⁹/L<75×109/L using a single starting dose level of 75 mg.

### Inclusion Criteria

Subjects are eligible to be included in the Phase 2 study if all the following criteria apply:

1. Male or female ≥18 years of age.

2. Confirmed diagnosis of MDS in accordance with the 2016 World Health Organization (WHO) criteria (Arber et al., Blood. 2016; 127: 2391-405) with the exception of patients with a diagnosis of therapy-related MDS or disease with the del 5q abnormality. Patients with secondary MDS or patients with MDS with isolated del(5q) are not eligible. Patients with MDS/MPN overlap are not eligible, but patients with MDS/MPN with ring sideroblasts and thrombocytosis [MDS/MPN-RS-T] are eligible.

3. IPSS-R classification of very low, low, or intermediate risk disease (Greenberg et al., Blood. 2012; 120: 2454-65).

4. Patients with ring sideroblasts ≥15% of erythroid precursors in bone marrow or >5% ring sideroblasts and presence of SF3B1 mutation should have been previously treated with luspatercept.

5. Patients with ring sideroblast <15% of erythroid precursors in bone marrow and no SF3B1 mutation should be refractory or intolerant to prior ESA treatment, or be unlikely to benefit from ESA therapy (endogenous erythropoietin levels >200 U/L).

6. Must be transfusion dependent requiring an average of 2-6 units/8 weeks of packed RBCs (pRBCs) during the 16 weeks prior to enrollment, with no consecutive 8-week period that was RBC transfusion-free during these 16 weeks.

7. Acceptable laboratory assessments obtained within 28 days prior to the first dose of study medication:
a. Absolute neutrophil count (ANC) ≥750×10⁶/L in the absence of growth factors within the previous 14 days.
b. Platelet count ≥50×10⁹/L in the absence of platelet transfusion(s) or platelet stimulating agents.
c. Peripheral blood and bone marrow blast count <5%
d. Aspartate aminotransferase (AST) and alanine aminotransferase (ALT)≥2.5×upper limit of normal (ULN).
e. Serum direct bilirubin <2.0×ULN.
f. Calculated or measured creatinine clearance (CrCl) of ≥30 mL/min.
8. Eastern Cooperative Oncology Group (ECOG) performance status of ≤2
9. Patients must be able to swallow solid forms of drugs.
10. Both male and female patients and partners of patients, with reproductive potential, must agree to use at least one highly effective contraceptive method (preferably low user dependency contraception methods, in particular when contraception is introduced as a result of participation in a clinical study) while on study therapy and for 94 days after the last dose of study drug for male patients and male partners of female patients, and for 184 days after the last dose of study drug for female patients and female partners of male patients. NOTE: Patients may consider seeking information from the study investigator regarding donation and cryopreservation of germ cells prior to treatment. Male patients should be informed of the risk of testicular toxicity and provided with adequate advice regarding sperm preservation.

### Alternative Inclusion Criteria

Subjects are eligible to be included in the Phase 2 study if all the following criteria apply:

1. Patient must be ≥18 years of age and the legal age of consent in the jurisdiction in which the study is taking place at the time of signing the informed consent form (ICF).

2. Patient has a documented diagnosis of MDS according to the World Health Organization (WHO) 2022 classification (Arber et al., Blood. 2016; 127: 2391-405) that meets IPSS-R classification (Greenberg et al., Blood. 2012; 120: 2454-65) of very low, low, or intermediate risk disease with score ≤3.5, and has <5% blasts in bone marrow.

3. Patient requires RBC transfusions, as documented by the following criteria:
3a. Average transfusion requirement of 2 to 6 units/8 weeks of RBCs confirmed for a minimum of 16 weeks immediately preceding start of treatment,
3b. Hgb levels at the time of or within 7 days prior to administration of an RBC transfusion must have been ≤9.5 g/dL in order for the transfusion to be counted towards meeting eligibility criteria, and
   Note: RBC transfusions administered for elective surgery, infections or bleeding events will not qualify as a required transfusion for the purpose of meeting eligibility criteria.
3c. No consecutive 56-day period that was RBC transfusion-free during the 16 weeks immediately preceding start of treatment.

4. Acceptable laboratory assessments obtained within 28 days prior to the first dose of study medications:
4a. Absolute neutrophil count (ANC) ≥750×10⁹/L in the absence of growth factors within the 14 days prior to the start of study treatment,
4b. PLT count ≥75×10⁹/L in the absence of PLT transfusion(s) or PLT stimulating agents within the 14 days prior to the start of study treatment. Patients with PLT count ≥75×10⁹/L are eligible to be included in pelabresib monotherapy in 2 dose levels cohorts (Stage 1 & Stage 2).
4c. PLT count ≥50 and <75×10⁹/L in the absence of PLT transfusion(s) or PLT stimulating agents within the 14 days prior to the start of study treatment. Patients with PLT count ≥50 and <75×10⁹/L are eligible only for safety cohort (pelabresib monotherapy in Stage 2),
4d. Calculated or measured creatinine clearance of ≥45 mL/min. (using Cockroft and Gault (Nephron 1976; 16(1):31-41. doi: 10.1159/000180580),
4e. Bilirubin ≤1.5×upper limit of normal (ULN), and
4f. Isolated elevation of aspartate aminotransferase (AST) and/or alanine aminotransferase (ALT)≤3×ULN of the local reference interval (≤5×if the elevation can be ascribed to liver involvement, e.g., presence of MDS with liver cirrhosis).

5. Patient has Eastern Cooperative Oncology Group (ECOG) score of 0, 1, or 2.

6. Willingness to avoid pregnancy or fathering children based on the criteria below:
Male patients and their female partners of childbearing potential must agree to take appropriate precautions to avoid fathering children (with at least 99% certainty) from Screening through 94 days after the last dose of pelabresib. Additionally, male patients must refrain from donating sperm during this period. Permitted methods that are at least 99% effective in preventing pregnancy should be communicated to the patients and their understanding confirmed.
Women of childbearing potential (WOCBP) must have a negative serum pregnancy test at Screening and must agree to take appropriate precautions to avoid pregnancy (with at least 99% certainty) from Screening until the end of relevant systemic exposure (i.e., through 184 days after the last dose of pelabresib). They must also agree to regular urine pregnancy testing through the study treatment period and monthly pregnancy testing up to 184 days after the last dose of pelabresib. They must also refrain from breastfeeding and donating oocytes during the course of study and for 184 days after the last dose of pelabresib. Permitted methods that are at least 99% effective in preventing pregnancy should be communicated to the patients and their understanding confirmed.
Women without childbearing potential (i.e., surgically sterile with a hysterectomy and/or bilateral oophorectomy OR postmenopausal with ≥12 months of amenorrhea without an alternative medical cause) are eligible.

### Exemplary Cohorts for Pelabresib Monotherapy Treatment

Patients in monotherapy cohorts are eligible to be included in the study only if all the criteria in the alternative exclusion criteria set forth above are met, and the following additional criteria are fulfilled:

7. According to investigator's assessment, patients are refractory, intolerant, or unlikely to benefit from at least one prior treatment, except of RBC transfusions.

8. Ring sideroblast (RS) status at baseline (with RS+ defined as RS≥15% of erythroid precursors in bone marrow or ≥5% (but <15%) if SF3B1 mutation is present). Up to 40% of patients randomized will be RS+. Enrollment of the total of the 60 patients randomized into 2 dose level in Stage 1 and Stage 2 will be capped according to the RS status.
Note: This is not applicable to the patients enrolled into pelabresib monotherapy with PLT count ≥50-<75×10⁹/L (safety cohort; n=10).

### Exclusion Criteria

Subjects are excluded from the Phase 2 study if all the following criteria apply:

### A. Medical Conditions

1. Patients with secondary MDS or patients with the del 5q abnormality are not eligible. Patients with MDS/myeloproliferative neoplasm (MPN) overlap are not eligible, with the exception of MDS/MPN with ring sideroblasts and thrombocytosis [MDS/MPN-RS-T].

2. Known clinically significant anemia due to iron, vitamin B12, or folate deficiencies, or autoimmune or hereditary hemolytic anemia.

3. Have current known active or chronic infection with HIV, hepatitis B, or hepatitis C. Screening of patients with serologic testing for these viruses is not required. However, patients who have a past history of viral hepatitis or in whom there is a current suspicion of viral hepatitis should have serologic testing for hepatitis B and hepatitis C performed to determine whether there is any current evidence for ongoing infection with these viruses.

4. Have an active clinically uncontrolled infection significant flare of a chronic infection. Patients will not be eligible for enrollment until recovery to ≤Grade 1 for at least 2 weeks prior to the first dose of study drug. Testing for COVID-19 is not mandatory during the screening for this study. However, based on the local epidemiologic situation and each patient's individual COVID-19 exposure risk and/or vaccination status, investigators should consider testing and in the case of COVID-19 positivity consider delaying the start of the study treatment until the infection is resolved.

5. Patients with certain gastrointestinal (GI) conditions that may, as per Investigator judgement, interfere with their ability to swallow solid forms of drugs and the absorption of oral drugs (e.g., inflammatory bowel disease [i.e., ulcerative colitis, Crohn's disease or celiac disease) are not eligible for participation in the study].

6. Have impaired cardiac function or clinically significant cardiac diseases, including any of the following:
Acute myocardial infarction or unstable angina pectoris ≤6 months prior to starting study drug.
QTcF>500 msec on the screening ECG.
New York Heart Association Class III or IV congestive heart failure.
Uncontrolled clinically significant cardiac arrhythmia (patients with rate-controlled atrial fibrillation are not excluded) Note that patients with a history of coronary artery disease and revascularization are not excluded.

7. Have ongoing uncontrolled hypertension (resting systolic blood pressure >160 mmHg and resting diastolic blood pressure >100 mmHg) despite maximal treatment with at least 2 anti-hypertensive agents.

8. Have ongoing uncontrolled diabetes (HbA1c ≥9%) despite maximal treatment with oral and/or injectable anti-hyperglycemic agents.

9. Have a history of a concurrent or second malignancy except for adequately treated local basal cell or squamous cell carcinoma of the skin, cervical carcinoma in situ, superficial bladder cancer, asymptomatic prostate cancer without known metastatic disease and with no requirement for therapy or requiring only hormonal therapy and with normal prostate-specific antigen for ≥1 year prior to randomization, adequately treated Stage 1 or 2 cancer currently in complete remission, or any other cancer that has been in complete remission for ≥3 years.

10. Have any other concurrent severe and/or uncontrolled concomitant medical condition that in the opinion of the Investigator could compromise participation in the study or analysis of study data. This includes but is not limited to clinically significant pulmonary disease or neurological disorders.

### B. Prior/Concomitant Therapy

11. Prior treatment with a hypomethylating agent (HMA) (azacitidine, decitabine)

12. Prior treatment with lenalidomide

13. Prior treatment with an investigational agent for MDS.

14. Prior immunosuppressive therapy (e.g., anti-thymocyte globulin [ATG], cyclosporine-based regimens, etc.) for MDS

15. Prior treatment with a BET inhibitor

16. Treatment with a strong CYP3A4 inhibitor or inducer within 7 days prior to the first dose of study drug including St. John's wort. Initiation of treatment or concomitant use of a strong CYP3A4 inhibitor or inducer during study treatment is prohibited.

17. Prior hematopoietic stem cell transplant

18. Patients who have started iron chelation therapy within 56 days prior to Cycle 1, Day 1 (C1D1) are not eligible for participation in the study, except for those patients who were on a stable or decreasing dose for ≥8 weeks prior to enrollment

### C. Other Exclusions

19. Females who are breastfeeding or pregnant as documented by a highly-sensitive serum β-hCG pregnancy test consistent with pregnancy, obtained within 72 hours prior to the first dose of study drug. Female patients of non-childbearing potential (post-menopausal for more than 1 year; underwent permanent sterilization by hysterectomy, or bilateral salpingectomy, or bilateral oophorectomy) do not require a serum pregnancy test.

20. Are unwilling or unable to comply with this study protocol or study requirements.

### Alternative Exclusion Criteria

Patients are excluded from the study if any of the following criteria apply:

### A. Medical Conditions

1. Patients with secondary MDS, with MDS/MPN overlap with the exception of MDS/MPN with RS and thrombocytosis).
2. Patients with known clinically significant anemia due to iron, vitamin B12, or folate deficiencies, known autoimmune or hereditary hemolytic anemia, clinically significant bleeding drug induced anemia, known hypothyroidism.
3. Patients with current known active or chronic infection with HIV, hepatitis B, or hepatitis C. Screening of patients with serologic testing for these viruses is not required. However, patients who have a past history of viral hepatitis or in whom there is a current suspicion of viral hepatitis should have serologic testing for hepatitis B and hepatitis C performed to determine whether there is any current evidence for ongoing infection with these viruses.
4. Patients with an active clinically uncontrolled infection significant flare of a chronic infection. Patients will not be eligible for enrollment until recovery to ≤Grade 1 for at least 2 weeks prior to the first dose of study treatment. Testing for COVID-19 is not mandatory during the screening for this study. However, based on the local epidemiologic situation and each patient's individual COVID-19 exposure risk and/or vaccination status, investigators should consider testing and in the case of COVID-19 positivity consider delaying the start of the study treatment until the infection is resolved.
5. Patients with certain GI conditions that may, as per Investigator judgement, interfere with their ability to swallow solid forms of drugs and the absorption of oral drugs (e.g., inflammatory bowel diseases or celiac disease).
6. Patients with impaired cardiac function or clinically significant cardiac diseases, including any of the following:
   6a. Acute myocardial infarction or unstable angina pectoris ≤6 months prior to starting study treatment,
   6b. The corrected QT interval by Fridericia (QTcF) >470 msec on the screening electrocardiogram (ECG) (QTcF interval is not relevant in patients with pacemaker-controlled arrythmia),
   6c. New York Heart Association Class III or IV congestive heart failure, or
   6d. Uncontrolled clinically significant cardiac arrhythmia (patients with rate-controlled atrial fibrillation are not excluded).
      Note: patients with a history of coronary artery disease and revascularization are not excluded.
7. Patients with ongoing uncontrolled hypertension (resting systolic blood pressure >160 mmHg and resting diastolic blood pressure >100 mmHg).
8. Patients with ongoing uncontrolled diabetes (glycated hemoglobin [HbA1c]≥9%).
9. Patients with history of a concurrent or second malignancy except for adequately treated local basal cell or squamous cell carcinoma of the skin, cervical carcinoma in situ, superficial bladder cancer, asymptomatic prostate cancer without known metastatic disease and with no requirement for therapy or requiring only hormonal therapy and with normal prostate-specific antigen for ≥1 year prior to start of treatment, adequately treated Stage 1 or 2 cancer currently in complete remission, or any other cancer that has been in complete remission for ≥3 years.
10. Patients with any other concurrent severe and/or uncontrolled concomitant medical condition that in the opinion of the Investigator could compromise participation in the study or analysis of study data. This includes but is not limited to clinically significant pulmonary disease or neurological disorders.

### B. Concomitant Therapy

11. Prior treatment with an HMA (azacytidine, decitabine) with last dose <8 weeks prior to the first dose of study treatment. Patients may be enrolled at the investigator's discretion. (The last dose must be ≥8 weeks prior to the first dose of study treatment.)
12. Prior treatment with immune-modulatory drugs ([IMiDs] such as lenalidomide) for conditions other than del(5q) abnormality. Patients may be enrolled at the investigator's discretion. (The last dose of IMiD must be ≥8 weeks prior to the first dose of study treatment.)
13. Concomitant treatment with an investigational agent within 28 days (or 5 half-lives, whichever is longer) prior to the first dose of study treatment.
14. Prior immunosuppressive therapy (e.g., anti-thymocyte globulin, cyclosporine-based regimens, etc.) for MDS.
15. Prior treatment with a BET inhibitor.
16. Treatment with a strong CYP3A4 inhibitor or inducer within 7 days prior to the first dose of study treatment, including St. John's wort. Initiation of treatment or concomitant use of a strong CYP3A4 inhibitor or inducer during study treatment is prohibited.
17. Prior hematopoietic stem cell transplant.
18. Patients who have started iron chelation therapy within 56 days prior to first dose of study treatment are not eligible for participation in the study, except for those patients who were on a stable or decreasing dose for ≥8 weeks prior to first dose of study treatment.
19. Prior treatment with androgens within 8 weeks of the first dose of study treatment, except for treatment of hypogonadism.
20. Concomitant treatment with RBC hematopoietic growth factors within 28 days of the first dose of study treatment.

### C. Other Exclusions

21. Not capable of giving signed informed consent which includes compliance with the requirements and restrictions listed in the ICF and in this protocol.
22. Patient is unwilling and unable to adhere to the study visit schedule and other protocol requirements. Patients who are legally institutionalized, or under judicial protection.
23. Concurrent enrollment in another interventional clinical trial.
24. History of hypersensitivity to any of the study treatments or its excipients or to drugs of similar chemical class.
25. Female patients who are breastfeeding or pregnant or not on adequate contraceptive therapy as specified in inclusion criteria.
26. Any male patient who does not agree to use contraception during the treatment period and for at least 94 after the last dose of study treatment (if they have a heterosexual partner who is a woman of childbearing potential) and who does not refrain from donating sperm during this period.
27. Patient with history of cerebrovascular accident (including ischemic, embolic, and hemorrhagic cerebrovascular accident), transient ischemic attack, deep venous thrombosis (including proximal and distal), pulmonary or arterial embolism, arterial thrombosis or other venous thrombosis within 6 months prior to randomization Note: prior superficial thrombophlebitis is not an exclusion criterion.

### Primary Endpoint

The primary endpoint for the studies include eight-week RBC transfusion independence (TI), defined as the absence of RBC transfusion during any consecutive 56-day period after the start of treatment.

### Secondary Endpoints

Secondary endpoints include the following:
12-week RBC TI, defined as the absence of RBC transfusion during any consecutive 84-day period after the start of treatment;
Time to 8-week RBC TI, defined as time from the first dose of study drug until the first onset of 56-day RBC TI;
Duration of 8-week RBC-TI, defined as time between the last RBC transfusion before an achievement of 8-week RBC TI and the first RBC transfusion after the achievement (if there are multiple achievements, the one associated with the longest duration will be recognized);
Hbg Response, defined as a mean Hgb increase of ≥1.0 g/dL from baseline over any 8-week RBC transfusion-free period post baseline;
Erythroid Response (mHI-E), defined as an RBC transfusion reduction of ≥4 units from baseline during any 8-week period post baseline (for patients with a baseline RBC transfusion burden of ≥4 units/8 weeks) or a mean Hgb increase of ≥1.5 g/dL from baseline over any 56-day RBC transfusion-free period post baseline (for patients with a baseline RBC transfusion burden of <4 units/8 weeks); and
Safety and tolerability will be assessed by the incidence of AEs and SAEs, and by evaluating changes in the vital signs, physical examinations and clinical laboratory values.

### Exploratory Endpoints

Exploratory endpoints include the following:
Time to 12-week RBC TI, defined as time from the first dose of study drug until the first onset of 84-day RBC TI.
Duration of 12-week TI, defined as time between the last RBC transfusion before an achievement of 12-week RBC TI and the first RBC transfusion after the achievement (if there are multiple achievements, the one associated with the longest duration will be recognized.)
Neutrophil Response (HI-N), defined as a relative increase of ≥100% and an absolute increase of >0.5×10⁹/L from baseline in neutrophil count at every assessment during any 8-week period post baseline, for patients with baseline neutrophil count of <1.0×10⁹/L.
Platelet Response (HI-P), defined as an absolute increase of ≥30×10⁹/L from baseline in platelet count (for patients with a baseline platelet count between 20×10⁹/L and 100×10⁹/L), or a platelet count of >20×10⁹/L and a relative increase of ≥100% from baseline (for patients with a baseline platelet count of <20×10⁹/L), at every assessment during any 8-week period post baseline.
PK of pelabresib as assessed by Cmax.
Target engagement is defined as gene expression changes in peripheral blood pre- and post-treatment.
PD effects on blood and bone marrow cells as assessed by including, but not limited to, effects on RBC and Mk progenitor populations, genes that regulation the RBC and Mk lineage differentiation.
Changes in inflammatory cytokines in pre- and post-treatment blood samples and cytokine transcription levels in bone marrow aspiration.
Changes of cytogenetic and mutational profiles in pre-treatment, during treatment, and at the end of treatment blood and/or bone marrow samples QoL, as assessed by EORTC-QLC-30 and FACT-An questionnaires. Change in HRQoL at Week 24 compared to baseline.

### Dose Modifications

In alternatives, pelabresib monohydrate will be administered at two starting dose levels (i.e. 75 mg and 150 mg) in patients with baseline platelet (PLT) counts ≥75×10⁹/L. Further clinical evaluations will be performed in patients with baseline PLT counts ≥50×109/L<75×10⁹/L using a single starting dose level of 75 mg. Pelabresib monohydrate will be administered QD for 14 consecutive days followed by a 7-day break, which is considered 1 cycle of treatment (1 cycle=21 days), until unacceptable toxicity or disease progression.

The pelabresib monohydrate dose may be increased in increments of 25 mg QD, no more often that once every 2 cycles, up to a maximum of 175 mg or 200 mg QD provided the following are observed:
Platelet count is ≥75×10⁹/L
Absolute neutrophil count (ANC) is ≥750×10⁶/L in the absence of growth factors
No bleeding incident(s)
No ≥Grade 3 AE(s) attributed to pelabresib

Guidance for dose modifications in the continuous treatment period, including reduction and/or hold of pelabresib treatment, due to platelet count decrease are provided in Table 2 and due to other toxicities are provided in Table 1.

TABLE 1
Dose Modification Table for Toxicities in MDS Patients

| | |
|---|---|
| Toxicity | Actions Required |

Hematology

| | |
|---|---|
| Grade 4 neutropenia | Hold pelabresib treatment for up to 28 days. Repeat |
| (ANC < 0.5 × 10⁹/L) | CBC at least twice weekly until resolution to ≤Grade |
| | 3. |
| | Once resolved to ≤Grade 3, restart pelabresib |
| | monohydrate administration (reduced by 25 mg QD). |
| | If 75 mg QD was the prior dose, restart treatment at |
| | 75 mg QD. |
| Reduction in platelet count | See Table 2 |

Hepatic

| | |
|---|---|
| ≥Grade 3 direct bilirubin (>3 × | Hold pelabresib treatment up to 28 days. |
| ULN) | Repeat direct bilirubin at least weekly until resolution |
| | to ≤Grade 1 |
| | Once resolves to ≤Grade 1, restart pelabresib |
| | pelabresib monohydrate administration reduced by 25 |
| | mg QD. If 75 mg QD was the prior dose, restart |
| | treatment at 75 mg QD |
| ≥Grade 3 ALT/AST (>5 × | Hold pelabresib treatment up to 28 days. |
| ULN) in patients who enroll | Repeat serum transaminases at least weekly until |
| with ≤Grade 1 ALT/AST | resolved to ≤Grade 1 or baseline. |
| OR | Once resolves to ≤Grade 1 or baseline, restart |
| Tripling of ALT/AST in | pelabresib monohydrate administration reduced by 25 |
| patients who enroll with Grade | mg QD. If 75 mg QD was the prior dose, restart |
| 2 ALT/AST | treatment at 75 mg QD |
| ≥Grade 2 total bilirubin | Hold pelabresib treatment up to 28 days |
| (>1.5 × ULN or >3 × ULN for | If another cause of increased liver function tests is |
| patients with Gilbert syndrome | identified, restart pelabresib monohydrate |
| not attributable to Gilbert | administration at same dose once resolved to ≤Grade |
| syndrome) AND ≥Grade 2 | 1 or baseline |
| ALT/AST (>3 × ULN) in | In the absence of biliary obstruction or other potential |
| patients who enroll | causes deemed responsible for the concurrent |
| with ≤Grade 1 ALT/AST | elevation of direct bilirubin and ALT/AST |
| OR | permanently discontinue pelabresib treatment. |
| ≥Grade 2 total bilirubin | |
| (>1.5 × ULN or >3 × ULN for | |
| patients with Gilbert syndrome | |
| not attributable to Gilbert | |
| syndrome) AND tripling of | |
| ALT/AST in patients who | |
| enroll with Grade 2 ALT/AST | |

Gastrointestinal

| | |
|---|---|
| ≥Grade 3 vomiting, diarrhea or | Treat with optimal supportive care as per institutional |
| Grade 3 nausea | guidelines (see Section 6.6.1 and Section 6.6.2) until |
| | resolution to ≤Grade 1. |
| | Hold pelabresib treatment up to 28 days. |
| | Patient should be contacted by investigator or study |
| | nurse daily until resolution to ≤Grade 1 or a decision |
| | is made to increase support (e.g., hospitalization). |
| | Resume pelabresib monohydrate administration in |
| | the presence of symptomatic prophylaxis treatment |
| | for GI condition, at same dose if duration is ≤72 |
| | hours, or resume with reduced by 25 mg QD (if 75 |
| | mg QD was the prior dose, restart treatment at 75 mg |
| | QD) if the duration is >72 hours or if hospitalization |
| | is required despite optimal supportive care |

Rash

| | |
|---|---|
| ≤Grade 2 | Provide supportive care. |
| Grade 3 | Provide supportive care. |
| | If resolution to ≤Grade 1 takes >72 hours, hold |
| | pelabresib treatment until resolution to ≤Grade 1. |
| | Resume pelabresib monohydrate administration with |
| | dose reduced by 25 mg/day. If 75 mg QD was the |
| | prior dose, restart treatment at 75 mg QD. |
| Grade 4 | Provide supportive care . . . |
| | Hold pelabresib treatment until resolution to ≤Grade |
| | 1. |
| | Resume pelabresib monohydrate administration with |
| | dose reduced by 25 mg/day. If 75 mg QD was the |
| | prior dose, restart treatment at 75 mg QD |

Other Non-Specified

| | |
|---|---|
| ≥Grade 2 that, in the opinion of | For Grade 2, consider dose reduction of pelabresib |
| the investigator, requires dose | monohydrate by 25 mg QD. |
| reduction | For Grades 3 and 4, HOLD pelabresib treatment up |
| | to 28 days. Evaluate at least once weekly until |
| | toxicity resolves to ≤Grade 2. Resume with dose of |
| | pelabresib monohydrate reduced by 25 mg QD. If 75 |
| | mg QD was the prior dose, restart treatment at 75 mg |
| | QD. Consult with company medical conitor prior to |
| | resuming of pelabresib in cases of toxicity of Grade 2 |
| | or any toxicity resolution with sequelae |
| Any toxicity caused by | Permanently discontinue pelabresib treatment if a |
| pelabresib that requires >28 | break of >28 days off is required unless there are |
| days hold | mitigating circumstances, such as evidence of clinical |
| | benefit. Reasons for extension must be discussed |
| | with and approved by the Company medical monitor. |

ALT = alanine aminotransferase;
ANC = absolute neutrophil count;
CBC = complete blood count;
ET = essential thrombocythemia;
ULN = upper limit of normal

In case a patient becomes infected with COVID-19 during the study, the Investigator should use their clinical judgment, in consultation with the Sponsor's medical monitor as needed, about study drug interruption, depending on the patient's symptoms, disease status, comorbidities, and concomitant medications. Pelabresib should be held for ≥Grade 3 infection. In cases of Grade 1-2 COVID-19 infection and/or COVID-19-related medical conditions, pelabresib may also be held per investigator clinical judgment and according to local and institutional standards of care.

TABLE 2
Pelabresib Dose Modification for MDS Patients for Reduction in Platelet Count

| Platelet Count on Day 1 of | Pelabresib Monohydrate Dose at Time of Platelet |
|---|---|
| any cycle | Decline^{a} |
| <lower limit of normal-75 × | No change |
| 10⁹/L (Grade 1) | |
| 50-<75 × 10⁹/L (Grade 2) | Consider dose reduction by 25 mg, depending on the |
| | change in platelet count. If 75 mg QD was the prior |
| | dose, continue treatment at 75 mg QD. |
| <50-25 × 10⁹/L (Grade 3) | HOLD^{b} treatment until PLT recovers to ≥50 × 10⁹/L |
| | If PLT recovers ≥50-<75 × 10⁹/L, restart treatment |
| | at a dose of 25 mg QD lower than the prior dose. If |
| | 75 mg QD was the prior dose, restart |
| | treatment at 75 mg QD. |
| | If PLT recovers ≥75 × 10⁹/L, and this is a first |
| | occurrence of grade 3 thrombocytopenia restart |
| | treatment at the prior dose |
| | For second occurrence of grade 3 thrombocytopenia |
| | regardless of platelet recovery, restart treatment at a |
| | dose of 25 mg QD lower than the prior dose. If 75 mg |
| | QD was the prior dose, restart treatment at 75 mg QD |
| <25 × 10⁹/L (Grade 4) | HOLD^{b} treatment until PLT recovers to ≥50 × 10⁹/L |
| | If PLT recovers ≥50-<75 × 10⁹/L, restart treatment |
| | at a dose 50 mg QD lower than the prior dose. If 75 |
| | mg QD was the prior dose, restart treatment at 75 mg |
| | QD. |
| | If PLT recovers ≥75 × 10⁹/L, restart treatment at a |
| | dose 25 mg QD lower than the prior dose. If 75 mg |
| | QD was the prior dose, restart treatment at 75 mg |
| | QD. |
| | If Grade 4 PLT recurs after restart of treatment and |
| | despite dose reduction to 75 mg daily, |
| | DISCONTINUE treatment |

BID = twice daily; ET = essential thrombocythemia; PLT = platelet; QD = once daily.
^{a}HOLD treatment if associated with bleeding. This clinical decision may be made at the discretion of the Investigator.
^{b}Check PLT at least weekly and resume dosing to maintain cycle structure. For example, if the toxicity occurred on day 8 of a cycle and resolved on day 15 of the same cycle, continue to hold dose during the remainder of the 7-day break (i.e. an additional 6 days) and resume dosing on day 1 of the next scheduled cycle. Discontinue treatment if HOLD for >28 days due to study treatment-related toxicity. Longer discontinuation times for mitigating circumstances, such as clinical benefit, must be discussed with and approved by the Company medical monitor.

### Dose Modifications for Toxicities

Guidance for dose modifications in the continuous treatment period, including reduction and/or hold of pelabresib, due to other toxicities are provided in Table 3 and due to PLT count decrease are provided in Table 4.

TABLE 3
Dose Modification Table for Toxicities in Myelodysplastic
Syndrome Patients in the Pelabresib Monotherapy Cohort

| | |
|---|---|
| Toxicity | Actions Required |

Anemia

| | |
|---|---|
| ≥Grade 3 anemia | Blood transfusion should be considered for severe |
| (Hgb <8.0 g/dL) | (Hgb <8.0 g/dL) and/or symptomatic anemia. |
| | No pelabresib dose reduction or interruption is needed for |
| | anemia, dose increase is allowed if other criteria in are met. |

Hematology

| | |
|---|---|
| Grade 4 neutropenia | Hold pelabresib treatment for up to 35 days. Repeat CBC at |
| (ANC <0.5 × 10⁹/L) | least twice weekly until resolution to ≤Grade 3. |
| | Once resolved to ≤Grade 3, restart pelabresib monohydrate |
| | administration (reduced by 25 mg QD or at 75 mg QD if |
| | 75 mg QD was the prior dose). |
| Reduction in PLT count | See Table 4 |

Hepatic

| | |
|---|---|
| ≥Grade 3 direct bilirubin | Hold pelabresib treatment up to 35 days. |
| (>3 × ULN) | Repeat direct bilirubin at least weekly until |
| | resolution to ≤Grade 1. |
| | Once resolves to ≤Grade 1, restart pelabresib monohydrate |
| | administration reduced by 25 mg QD. If 75 mg QD was the |
| | prior dose, restart treatment at 75 mg QD. |
| ≥Grade 3 ALT/AST (>5 × | Hold pelabresib treatment up to 35 days. |
| ULN) in patients who enroll | Repeat serum transaminases at least weekly |
| with ≤Grade 1 ALT/AST | until resolved to ≤Grade 1 or baseline. |
| OR | Once resolves to ≤Grade 1 or baseline, restart pelabresib |
| Tripling of ALT/AST in | monohydrate administration reduced by 25 mg QD. If |
| patients who enroll with | 75 mg QD was the prior dose, restart treatment at 75 mg |
| Grade 2 ALT/AST | QD. |
| ≥Grade 2 total bilirubin | Hold pelabresib treatment up to 35 days. |
| (>1.5 × ULN or >3 × ULN | If another cause of increased liver function tests is |
| for patients with Gilbert | identified, restart pelabresib monohydrate administration at |
| syndrome not attributable to | same dose once resolved to ≤Grade 1 or baseline. |
| Gilbert syndrome) | In the absence of biliary obstruction or other potential |
| AND ≥Grade 2 ALT/AST (>3 × | causes deemed responsible for the concurrent elevation of |
| ULN) in patients who enroll | direct bilirubin and ALT/AST permanently discontinue |
| with ≤Grade 1 ALT/AST | pelabresib. |
| OR | |
| ≥Grade 2 total bilirubin | |
| (>1.5 × ULN or >3 × ULN | |
| for patients with Gilbert | |
| syndrome not attributable to | |
| Gilbert syndrome) AND | |
| tripling of ALT/AST in | |
| patients who enroll with | |
| Grade 2 ALT/AST | |

Infection

| | |
|---|---|
| ≥Grade 3 | Provide supportive care. |
| | Hold pelabresib treatment until resolution to ≤G1 or for up |
| | to 35 days, whichever occurs first. |
| | Resume pelabresib monohydrate administration with dose |
| | reduced by 25 mg/day. If 75 mg QD was the prior dose, |
| | restart treatment at 75 mg QD at the next visit. |
| Grade 4 | Provide supportive care. |
| | Hold pelabresib treatment until infection resolves to |
| | Grade ≤1. |
| | Resume pelabresib monohydrate administration with dose |
| | reduced by 25 mg/day. If 75 mg QD was the prior dose, |
| | restart treatment at 75 mg QD at the next visit. |

Gastrointestinal

| | |
|---|---|
| ≥Grade 3 vomiting, | Treat with optimal supportive care as per institutional |
| diarrhea or Grade 3 nausea | guidelines until resolution to ≤Grade 1. |
| | Hold pelabresib treatment up to 35 days. |
| | Patient should be contacted by investigator or study nurse |
| | daily until resolution to ≤Grade 1 or a decision is made to |
| | increase support (e.g., hospitalization). |
| | Resume pelabresib monohydrate administration in the |
| | presence of symptomatic prophylaxis treatment for GI |
| | condition, at same dose if duration is ≤72 hours, or resume |
| | with reduced by 25 mg QD (if 75 mg QD was the prior |
| | dose, restart treatment at 75 mg QD) if the duration is >72 |
| | hours or if hospitalization is required despite optimal |
| | supportive care. |

Rash

| | |
|---|---|
| ≤Grade 2 | Provide supportive care. |
| Grade 3 | Provide supportive care. |
| | If resolution to ≤Grade 1 takes >72 hours, hold pelabresib |
| | treatment until resolution to ≤Grade 1. |
| | Resume pelabresib monohydrate administration with dose |
| | reduced by 25 mg/day. If 75 mg QD was the prior dose, |
| | restart treatment at 75 mg QD at the next visit. |
| Grade 4 | Provide supportive care. |
| | Retreatment is not permitted. Permanently discontinue |
| | pelabresib treatment. |

Other Non-specified

| | |
|---|---|
| ≥Grade 2 that, in the | For Grade 2, consider dose reduction of pelabresib |
| opinion of the investigator, | monohydrate administration by 25 mg QD. |
| requires dose reduction | For Grades 3 and 4, hold pelabresib treatment up to 35 days. |
| | Evaluate at least once weekly until toxicity resolves |
| | to ≤Grade 2. Resume with dose of pelabresib monohydrate |
| | administration reduced by 25 mg QD. If 75 mg QD was the |
| | prior dose, restart treatment at 75 mg QD. Consult with |
| | sponsor's Medical Monitor prior to resuming of pelabresib |
| | in cases of toxicity of Grade 2 or any toxicity resolution |
| | with sequelae. |
| Any toxicity caused by | Permanently discontinue pelabresib if a break of >35 days |
| pelabresib that requires >35 | off is required unless there are mitigating circumstances, |
| days hold | such as evidence of clinical benefit. Reasons for extension |
| | must be discussed with and approved by the sponsor's |
| | Medical Monitor. |
| Non-hematological toxicity | If the dose has been reduced for a non-hematological |
| | toxicity, dose increase cannot be instituted until the toxicity |
| | has resolved to ≤Grade 1 for at least 1 cycle. If |
| | reoccurrence, dose increases are prohibited. |
| | If the dose has been reduced due to Grade 4 non- |
| | hematologic toxicity, a subsequent dose increase is |
| | prohibited. |

ALT = alanine aminotransferase; ANC = absolute neutrophil count; CBC = complete blood count; ET = essential thrombocythemia; QD = once a day; ULN = upper limit of normal.

In case a patient becomes infected with COVID-19 during the study, the investigator should use their clinical judgment, in consultation with the sponsor's Medical Monitor as needed, about study treatment interruption, depending on the patient's symptoms, disease status, comorbidities, and concomitant medications. In cases of Grade 1-2 COVID-19 infection and/or COVID-19-related medical conditions, pelabresib may also be held per investigator clinical judgment and according to local and institutional standards of care.

TABLE 1
Pelabresib Dose Modification for Myelodysplastic
Syndrome Patients for Reduction in Platelet Count

| Platelet Count on Day | |
|---|---|
| 1 of any cycle | Pelabresib Monohydrate Dose at Time of Platelet Declinea |
| Grade 1 | No change |
| (<lower limit of | |
| normal-75 × 10⁹/L) | |
| Grade 2 | Consider dose reduction by 25 mg, depending on the change in |
| (50-<75 × 10⁹/L) | PLT count. If 75 mg QD was the prior dose, continue treatment |
| | at 75 mg QD. |
| Grade 3 | Holdb treatment until PLT recovers to ≥50 × 10⁹/L. |
| (<50-25 × 10⁹/L) | If PLT recovers ≥50-<75 × 10⁹/L, restart treatment at a dose of |
| | 25 mg QD lower than the prior dose at the next visit. If 75 mg |
| | QD was the prior dose, restart treatment at 75 mg QD at the next |
| | visit. |
| | If PLT recovers ≥75 × 10⁹/L, and this is a first occurrence of |
| | Grade 3 thrombocytopenia, restart treatment at the prior dose. |
| | For second occurrence of grade 3 thrombocytopenia regardless |
| | of PLT recovery, restart treatment at a dose of 25 mg QD lower |
| | than the prior dose. If 75 mg QD was the prior dose, restart |
| | treatment at 75 mg QD. |
| | After resuming treatment following a hold, check PLT weekly |
| | until resolution to Grade 2. |
| Grade 4 | Holdb treatment until PLT recovers to ≥50 × 10⁹/L. |
| (<25 × 10⁹/L) | Pelabresib dose reduced by 50 mg QD, except if the dose was |
| | 75 mg or 100 mg QD at the time of the hold, then restart at |
| | 50 mg QD (increase to 75 mg QD until Grade ≤2 PLT count is |
| | maintained for at least 2 cycles). |
| | If PLT recovers ≥75 × 10⁹/L, restart treatment at a dose 25 mg |
| | QD lower than the prior dose. If 75 mg QD was the prior dose, |
| | restart treatment at 75 mg QD. |
| | If Grade 4 PLT recurs after restart of treatment and despite dose |
| | reduction to 75 mg daily, discontinue treatment. |
| | After resuming treatment following a hold, check PLT weekly |
| | until resolution to Grade 2. |

BID = twice daily; ET = essential thrombocythemia; PLT = platelet; QD = once daily.
aHold treatment if associated with bleeding. This clinical decision may be made at the discretion of the Investigator.
bCheck PLT at least weekly and resume dosing to maintain cycle structure. For example, if the toxicity occurred on day 8 of a cycle and resolved on day 15 of the same cycle, continue to hold dose during the remainder of the 7-day break (i.e., an additional 6 days) and resume dosing on day 1 of the next scheduled cycle. Discontinue treatment if held for >35 days due to study treatment-related toxicity. Longer discontinuation times for mitigating circumstances, such as clinical benefit, must be discussed with and approved by the Constellation Medical Monitor.

Re-Escalation of Pelabresib after Dose Reduction for Toxicity

Patients who have had a dose reduction or dose hold of pelabresib because of an AE may increase the dose of pelabresib as follows:
If the indicated toxicity in Table 3 resolves to the specified grade (with exceptions noted below) for at least 1 cycle, the dose level may be upward titrated 1 dose level higher per cycle (25 mg/day). This can be repeated until the original dose level (defined as the dose level before receiving the downwards titration) is reached.
If a patient experiences Grade 4 neutropenia and the toxicity resolves (ANC is >0.75×10⁹/L) for at least 1 cycle, the dose level may be titrated upwards 1 dose level per cycle.
If a patient experiences any grade thrombocytopenia, all dose modifications must be made per Table 4. Re-escalation decisions must be made in accordance to criteria defined in for pelabresib.
If the dose has been reduced for a non-hematologic toxicity, a dose increase cannot be instituted until the toxicity has resolved to Grade ≤1 for at least 1 cycle. If the same toxicity recurs after dose re-escalation, further dose increases are prohibited.
If the dose is reduced due to Grade 4 non-hematologic toxicity, a subsequent dose increase is prohibited.

If these criteria are met, the pelabresib monohydrate dose can be increased in 25 mg QD increments, not more frequently than once every 2 cycles, to a maximum dose of 125 mg QD.

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating lower-risk myelodysplastic syndrome (LR-MDS) in a subject in need thereof comprising administering to the subject a therapeutically effective amount of pelabresib, or a pharmaceutically acceptable salt thereof.

2. The method of claim 1, wherein the subject is administered a therapeutically effective amount of pelabresib.

3. The method of claim 1 or 2, wherein the pelabresib is a hydrate.

4. The method of claim 1 or 3, wherein the pelabresib is a monohydrate.

5. The method of any one of claims 1 to 4, wherein the pelabresib is in crystalline form.

6. The method of any one of claims 1 to 5, wherein the pelabresib is crystalline Form A characterized by at least three, at least four, at least five, or by six x-ray powder diffraction peaks at 2Θ angles selected from 4.73°, 18.09°, 18.48°, 18.80°, 19.70°, and 25.17°.

7. The method of any one of claims 1 to 6, wherein the pelabresib is crystalline Form A characterized by at least three x-ray powder diffraction peaks at 2Θ angles selected from 4.730, 18.090, 18.480, 18.800, 19.700, and 25.170.

8. The method of any one of claims 1 to 7, wherein the pelabresib is crystalline Form A characterized by at least four x-ray powder diffraction peaks at 2Θ angles selected from 4.730, 18.090, 18.480, 18.800, 19.700, and 25.170.

9. The method of any one of claims 1 to 8, wherein the pelabresib is crystalline Form A characterized by at least five x-ray powder diffraction peaks at 2Θ angles selected from 4.73°, 18.09°, 18.48°, 18.80°, 19.70°, and 25.17°.

10. The method of any one of claims 1 to 9, wherein the pelabresib is crystalline Form A characterized by a x-ray powder diffraction peaks at 2Θ angles selected from 4.73°, 18.09°, 18.480, 18.800, 19.700, and 25.170.

11. The method of any one of claims 1 to 10, wherein the LR-MDS is low risk-MDS.

12. The method of any one of claims 1 to 11, wherein the LR-MDS is very low risk-MDS

13. The method of any one of claims 1 to 12, wherein the subject is anemic.

14. The method of any one of claims 1 to 13, wherein the subject is red blood cell transfusion dependent prior to treatment.

15. The method of claim 14, wherein the subject becomes transfusion independent during treatment.

16. The method of claim 14 or 15, wherein transfusion independent is characterized by the absence of a RBC transfusion for a period of about 8 consecutive weeks during treatment.

17. The method of claim 15, wherein transfusion independent is characterized by the absence of a RBC transfusion during any consecutive 56-day period after the start of treatment.

18. The method of any one of claims 1 to 17, wherein the subject experiences an improvement in hemoglobin levels during treatment.

19. The method of claim 18, wherein the improvement in hemoglobin is characterized as an increase in hemoglobin levels of about ≥1.0 g/dL during treatment.

20. The method of any one of claims 1 to 16, 18, and 19, wherein the subject becomes transfusion independent characterized by the absence of a RBC transfusion for a period of about 8 consecutive weeks during treatment and wherein the subject experiences an improvement in hemoglobin characterized as a mean hemoglobin increase of ≥1.0 g/dL during the transfusion independent period of about 8 consecutive weeks.

21. The method of any one of claims 1 to 16, 18, and 19, wherein the subject becomes transfusion independent characterized by the absence of a RBC transfusion for a period of about 8 consecutive weeks during treatment and wherein the subject experiences an Erythroid Response (mHI-E) defined as an RBC transfusion reduction of ≥4 units during the transfusion independent period of about 8 consecutive weeks.

22. The method of any one of claims 1 to 16, 18, and 19, wherein the subject becomes transfusion independent characterized by the absence of a RBC transfusion for a period of about 8 consecutive weeks during treatment and wherein the subject experiences an Erythroid Response (mHI-E) defined as a mean hemoglobin increase of ≥1.5 g/dL during the transfusion independent period of about 8 consecutive weeks.

23. The method of any one of claims 1 to 22, wherein the subject experiences a Neutrophil Response (HI-N) during treatment.

24. The method of any one of claims 1 to 23, wherein the subject experiences a Neutrophil Response (HI-N) during treatment characterized by a relative increase of ≥100% and an absolute increase of >0.5×10⁹/L from baseline in neutrophil count at every assessment during any consecutive 8-week period post baseline, for patients with baseline neutrophil count of <1.0×10⁹/L.

25. The method of any one of claims 1 to 24, wherein the subject has a platelet count of ≥75×10⁹/L prior to treatment.

26. The method of any one of claims 1 to 24, wherein the subject has a platelet count of <75×10⁹/L prior to treatment.

27. The method of any one of claims 1 to 24, wherein the subject has a platelet count of ≥50×10⁹/L<75×10⁹/L prior to treatment.

28. The method of any one of claims 1 to 27, wherein the subject experiences a Platelet Response (HI-P) during treatment.

29. The method of any one of claims 1 to 24, wherein the subject has a baseline platelet count between 20×10⁹/L and 100×10⁹/L and experiences a Platelet Response (HI-P) during treatment characterized by an absolute increase of ≥30×10⁹/L from baseline, at every assessment during any consecutive 8-week period post baseline.

30. The method of any one of claims 1 to 24, wherein the subject has a baseline platelet count of <20×10⁹/L and experiences a Platelet Response (HI-P) during treatment characterized by a relative increase of ≥100% from baseline, at every assessment during any consecutive 8-week period post baseline.

31. The method of any one of claims 1 to 30, wherein the subject is administered about 75 mg/day pelabresib.

32. The method of claim 31, wherein the subject has a baseline platelet count of <75×10⁹/L prior to treatment

33. The method of any one of claims 1 to 30, wherein the subject is administered about 125 mg/day pelabresib.

34. The method of any one of claims 1 to 30, wherein the subject is administered about 150 mg/day pelabresib.

35. The method of any one of claims 31, 33, and 34, wherein the subject has a baseline platelet count of ≥75×10⁹/L prior to treatment.

36. The method of any one of claims 1 to 30, wherein the subject is administered about 175 mg/day pelabresib.
